(19) United States
(12) Patent Application Publication
(10) Pub. No.: US 2002/0032418 A1
(43) Pub. Date: Mar. 14, 2002

(54) Device for securing ostomy pouches

(75) Inventor: Jason W. Iseke, Hauula, HI (US)

Correspondence Address: Martin E. Hsia, P.O. Box 939, Honolulu, HI, 96808-0939, US

(73) Assignee: REVOCABLE TRUST OF JASON W. ISEKE

(21) Appl. No.: 09/949,252

(22) Filed: Sep. 7, 2001

Related U.S. Application Data
(60) Provisional application No. 60/231,428, filed on Sep. 8, 2000.

(51) Int. Cl. A61F 5/44

(52) U.S. Cl. 604/338; 604/345

(57) ABSTRACT

A device for securing ostomy pouches over abdominal stomas. An intermediary flange (preferably plastic), with a central, circular aperture, is interposed between an optionally adhesive wafer and an ostomy pouch. Surrounding the circular aperture on the front of the flange is a single ridge adapter that interlocks with the ostomy pouch's double ridge adapter. Surrounding the circular aperture on the back of the flange is a double ridge adapter that interlocks with the wafer's single ridge adapter. A variation includes a flange (preferably plastic) with a central, circular aperture slightly larger than the outer ridge of the double ridge adapter, through which aperture the wafer and ostomy pouch interlock. An ostomy belt may be attached to attachment structures on the plastic flange to secure the optionally adhesive wafer to the body with the aperture over the stoma.

### BACKGROUND OF THE INVENTION

This invention relates to a device for securing ostomy pouches over abdominal stomas for the collection, storage and disposal of human bodily wastes.

Ostomy is a general term for an operation performed when the normal channels of elimination are dysfunctional or non-existent due to injury, disease, birth defect or other disorder. Stomas are surgically produced abdominal openings connecting one end of an internal organ to the skin surface. Stomas are used to discharge bodily wastes in the form of urine or feces into an external receptacle, such as an ostomy pouch. Different kinds of ostomies are named for the organ involved. The most common types of ostomies in intestinal surgery are ileostomies (connecting the small intestine to the skin) and colostomies (connecting the large intestine to the skin). A user of an ostomy pouch is sometimes referred to as an ostomate.

Conventionally, a pliable foundation component, commonly called a “wafer,” is adhered temporarily to the user's body with cement or other adhesive. Ostomy pouches are then removably and sealingly attached to the wafer, usually through interlocking ridges as described below. Cement is commonly used to attach the wafer to the user's body because it is the cheapest adhesive, but it may cause chemical burns. Further, when an adhesive is repeatedly compressed against the user's skin by a wafer, severe skin irritation may occur. Changing the wafer too frequently, or wearing one too long, may also cause skin irritations.

Furthermore, allergic contact dermatitis can result from a delayed allergic reaction to a substance in the wafer, such as the adhesive. Itching, redness, burning or stinging, and areas of moist, denuded skin are the most common types of reactions. Allergic contact dermatitis is treated by adjusting or changing the ostomy system to either eliminate or minimize contact between the skin and the wafer's adhesive. Because even “non-allergenic” tapes can occasionally cause this type of reaction, a non-adhesive, or optionally adhesive, method for attaching an ostomy pouch is the best solution.

Another problem ostomates encounter frequently is peristomal skin (skin surrounding the stoma) excoriation. This condition makes it uncomfortable or painful to touch the area or to apply an ostomy pouch when the skin is broken, and is described as skin that is red, weepy, possibly bleeding, and painful. Peristomal excoriation is caused by various factors, including wafer or ostomy pouch leakage, skin-stripping when too strong an adhesive is used, or when a wafer is removed in a rough manner.

Ostomates may also suffer from folliculitis, which is an inflammation of the hair follicles, in this case, around the stoma. Its appearance is described as redness at the base of the hair follicles around the stoma area. This condition is caused by removal of the hair from its follicle by aggressive adhesives, resulting in irritation or infection.

Irregularities in the skin's surface, resulting from rash and inflammation, combined with secretion of bodily fluids, often causes the bond between stoma and wafer to break and waste matter to be discharged. Peristomal skin must be protected from direct contact with the discharge because such discharge contains various substances that can lead to skin damage. For example, urostomy, or urinary diversion, stomas discharge urine, which changes to an ammonia-like substance when exposed to air and is capable of burning or corroding skin. Ileostomies and certain types of colostomies also discharge a caustic effluent containing digestive enzymes that can damage the skin. Moreover, skin irritations cannot be alleviated with creams, as the creams may cause adhesion problems with the wafers. Because of the discomfort, odor and unsanitary nature caused by these accidental discharges, there is a great need to modify the device and method by which ostomy pouches are attached to the body.

Showering also poses a problem for ostomates because they either need to remove the adhesive wafer, which may cause irritation if done too frequently, or they must forego cleaning the peristomal skin if they leave the wafer on, which may lead to infection.

Further, body movements during sleep can cause leakage if the ostomy pouch is not securely fastened to the wafer over the stoma.

Conventionally, the wafer is a disk with a central, circular aperture to be positioned over the stoma. A collar surrounds the disk, giving the wafer a square outline. Between the edge of the disk and the circular aperture is a circular single ridge adapter, concentric with the circular aperture. Wafers typically have a skin-contacting layer of a soft, pliant adhesive material on one side, which is protected until use by a sheet of paper or other suitable material. The other side usually has a collar of film or fabric. An example of one such wafer is disclosed in U.S. Pat. No. 4,738,257, in which the adhesive layer is of substantially uniform thickness. In recent years, contoured wafers have become available in which the layers are not of uniform thickness, as shown by, for example, U.S. Pat. Nos. 5,133,821, 4,876,748 and 5,609,585.

As noted above, the ostomy pouch collects waste matter that is discharged from an ostomate. Conventionally, a double ridge adapter, complementary to and interlockable with the wafer's single ridge adapter, surrounds a circular opening on one side of the ostomy pouch.

Several different ostomy pouch attachment mechanisms are available. For example, U.S. Pat. No. 4,460,363 issued to Steer describes a system by which an ostomy pouch can be coupled to an adhesive wafer that fits around the stoma while permitting the pouch to be removed without disturbing the wafer. U.S. Pat. No. 3,970,085 issued to Mersan describes a sealing mechanism connecting a base gasket that can be attached to the body using belts or adhesives to the ostomy pouch. U.S. Pat. No. 5,626,570 issued to Gallo describes an ostomy pouch belt for supporting and concealing an ostomy pouch.

Furthermore, hernia/ostomy support belts with a circular opening on the face of the belt provide support to an ostomy pouch user. After the ostomy pouch is adhered to the body with an adhesive wafer, the pouch is pulled through the opening in the support belt and worked through. As a result, the belt lies between the pouch and the adhesive wafer. Disadvantages of these inventions include the use of adhesives and increasing complexity of design.

With these considerations in mind, it is an object of this invention to provide a device of the character described that may be attached to the body without using skin-irritating adhesives.

It is a further object of this invention to provide a device that can be easily removed before showering.

It is a still further object of this invention to provide a device that can be readily used with conventional ostomy pouches, wafers and ostomy belts without further adaptation.

It is a still further object of this invention to provide ostomates with a comfortable method of securing the ostomy pouch to the stoma that holds the ostomy pouch in place during sleep.

### SUMMARY OF THE INVENTION

These and other objects are achieved by a device that includes an elongated flange having a central, circular aperture and wings on which attachment structures are located. In the present invention, these attachment structures are preferably slots. The flange is interposed between and sealingly engaged with both the wafer and the ostomy pouch. A belt can engage the slots to secure the wafer against the user's body.

In a first preferred embodiment, on the back, the flange has a double ridge adapter preferably identical to the conventional double ridge adapter of an ostomy pouch. On the front, the flange has a single ridge adapter preferably identical to the conventional single ridge adapter of a wafer. The flange is connected to a wafer by interlocking the wafer's single ridge adapter with the double ridge adapter mounted on the back of the flange so that the wafer interlocks with the flange, instead of with the ostomy pouch. An ostomy pouch is connected, on the side of the flange opposite the wafer, by interlocking the wafer's single ridge adapter mounted on the front of the flange with the ostomy pouch's double ridge adapter, so that the ostomy pouch interlocks with the flange, instead of with the wafer. A belt can engage the slots on the wings of the plastic flange to secure the wafer against a user's body. The belt can be adjusted so that it securely holds the wafer against the user's body. Thus, no adhesives are necessary to affix the wafer to the body.

In a second preferred embodiment, a flat plastic flange is provided with a central, circular aperture that is slightly larger than the ostomy pouch's double ridge adapter. The wafer's single ridge adapter then can be interlocked with the ostomy pouch's double ridge adapter through the flat plastic flange's central, circular aperture. As with the first preferred embodiment, a belt can engage the slots on the wings of the plastic flange to secure the wafer against a user's body. The belt can be adjusted so that it securely holds the wafer against the user's body, obviating the need for adhesives to affix the wafer to the body. This variation performs an identical function as the first embodiment, but provides a simpler design.

Other objects, features and advantages of the present invention will become more fully apparent from the following detailed description of the presently preferred embodiments for carrying out the invention and the accompanying drawings.

### BRIEF DESCRIPTION OF THE DRAWINGS

FIG. 1 is a front elevational view of a first presently preferred embodiment of the invention;

FIG. 2. is a rear elevational view of the embodiment of FIG. 1;

FIG. 3 is a side elevational view of the embodiment of FIG. 1;

FIG. 4 is a front elevational view of a second presently preferred embodiment of the invention;

FIG. 5 is a rear elevational view of the embodiment of FIG. 4;

FIG. 6 is a side elevational view of the embodiment of FIG. 4;

FIG. 7 is an exploded view of a first presently preferred embodiment of the invention and wafer shown from the side and an ostomy pouch shown in perspective;

FIG. 8 is a perspective exploded view of a second presently preferred embodiment of the invention; and

FIG. 9 is a front elevational view of an alternative embodiment of the present invention, substituting raised knobs for slots.

### DESCRIPTION OF A PRESENTLY PREFERRED EMBODIMENT

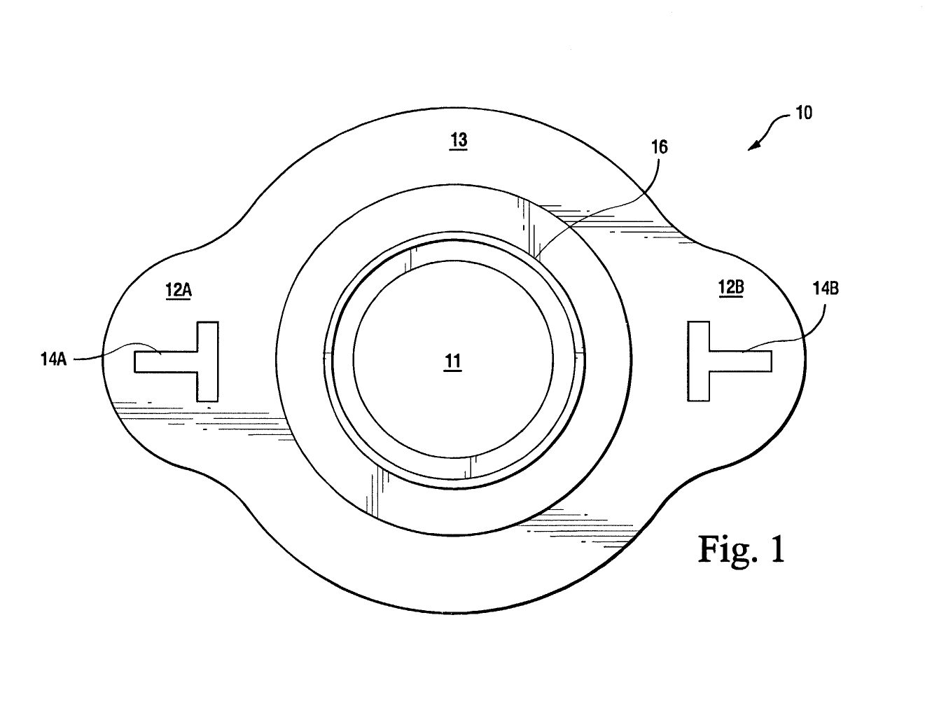

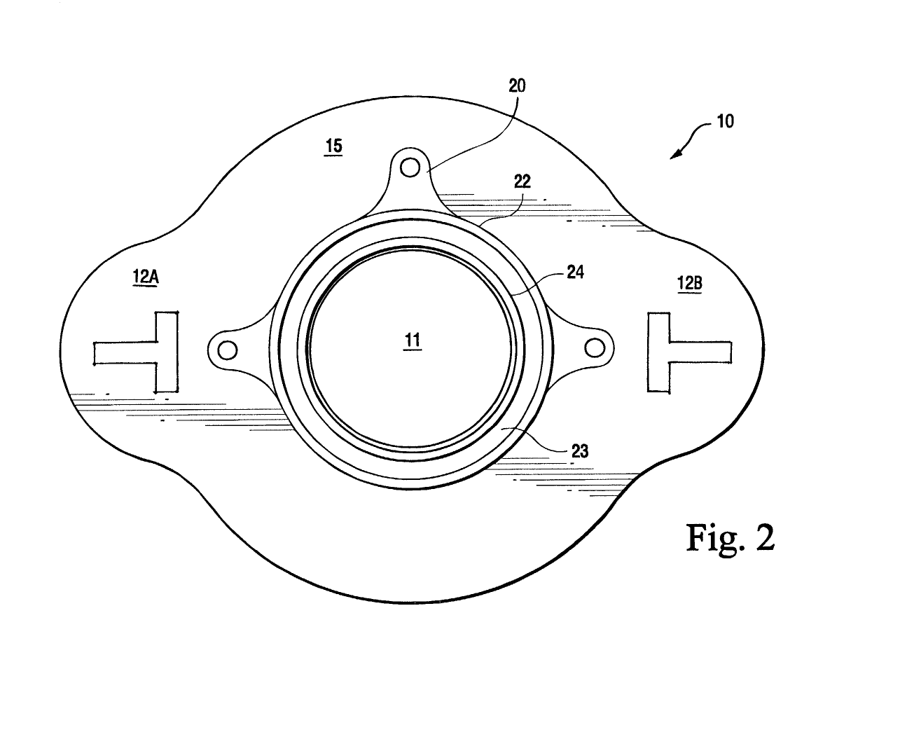

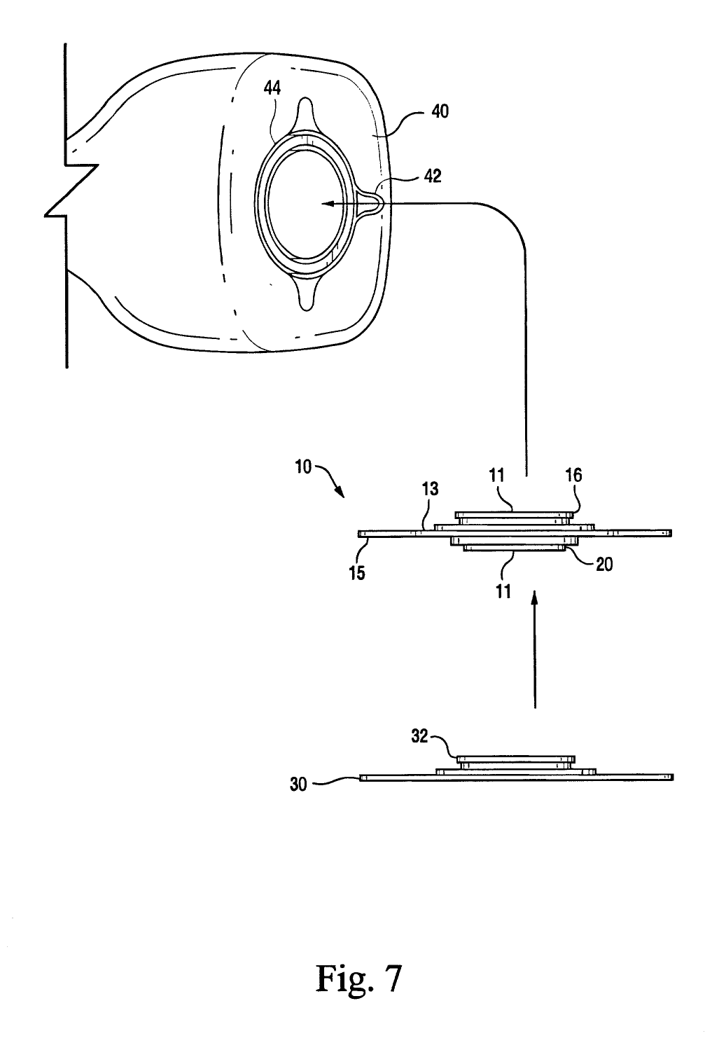

Referring to FIG. 1, shown is a front elevational view of a first preferred embodiment of the invention. A flat plastic flange 10 with a central, circular aperture 11 has wings 12A and 12B, on which attachment structures are located. In the present invention, these attachment structures are preferably slots 14A and 14B. The slots 14A and 14B are preferably “T”-shaped and can engagingly receive the attachment mechanisms at the ends of a conventional ostomy belt (not shown). The attachment mechanisms (which are conventionally tabs) at the ends of a conventional ostomy belt (not shown) can be received though the top of the “T”-shaped slots 14A and 14B, and slid along the slot to secure the belt. Concentrically encircling the circular aperture 11, is a coupling member comprising a single ridge adapter 16 (the male ring) provided on the front 13 of the flange 10 defining an external annular shoulder that interlocks with a conventional double ridge adapter 42 (the female ring) of an ostomy pouch 40 (see FIG. 7), and a double ridge adapter 20 (identical to the conventional double ridge adapter 42 (female ring) of an ostomy pouch 40) provided on the rear 15 of the flange 10, as shown in FIG. 2, which interlocks with the conventional single ridge adapter 32 of a wafer 30 (see FIG. 7) via an interlocking groove 23. The outer ridge 22 and inner ridge 24 of the double ridge adapter 20 define the interlocking groove 23.

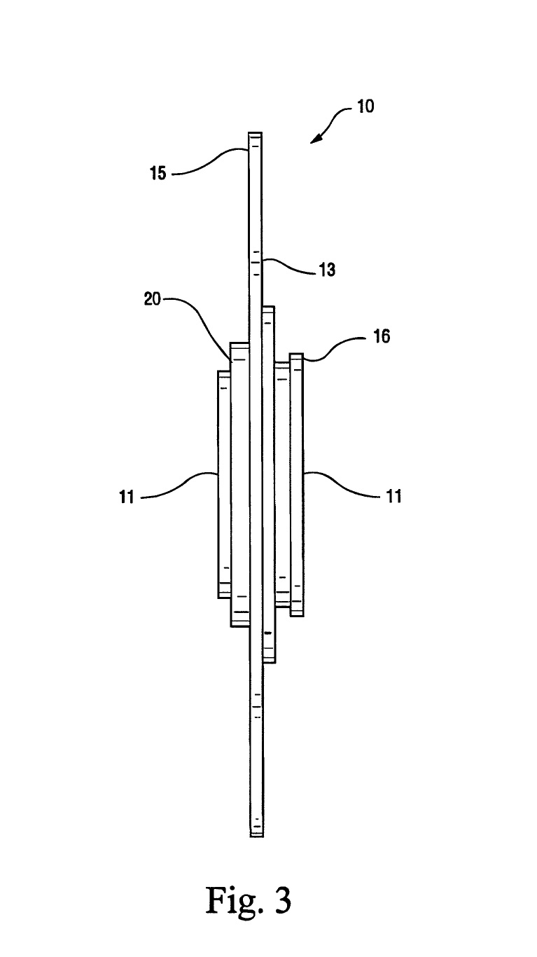

Referring to FIG. 3, a side elevational view of the coupling member of the first preferred embodiment of the flange 10 is shown. The single ridge adapter 16 surrounds the central, circular aperture 11, on the front 13 of the flange 10. The slots 14A and 14B are also located on wings 12A and 12B, respectively. Referring to FIG. 7, the front 13 of the flange 10 faces the ostomy pouch 40 when in use. The double ridge adapter 20 of the flat plastic flange 10 is located on the rear 15 of the flange 10. Referring also to FIG. 7, the rear 15 of the flange 10 faces the wafer 30 when in use.

Referring again to FIG. 7, in use, the flange 10 is connected to the wafer 30 by interlocking the wafer's single ridge adapter 32 with the double ridge adapter 20 mounted on the rear 15 of the flange 10. An ostomy pouch 40 is connected on the front 13 of the flange 10 (opposite the wafer 30) by interlocking the single ridge adapter 16 mounted on the front 13 of the flange 10 with the ostomy pouch's double ridge adapter 42. An ostomy belt (not shown) can then be attached to the flange 10 via the slots 14A, 14B on the wings 12A, 12B of the flange 10 to secure the wafer 30 against a user's body.

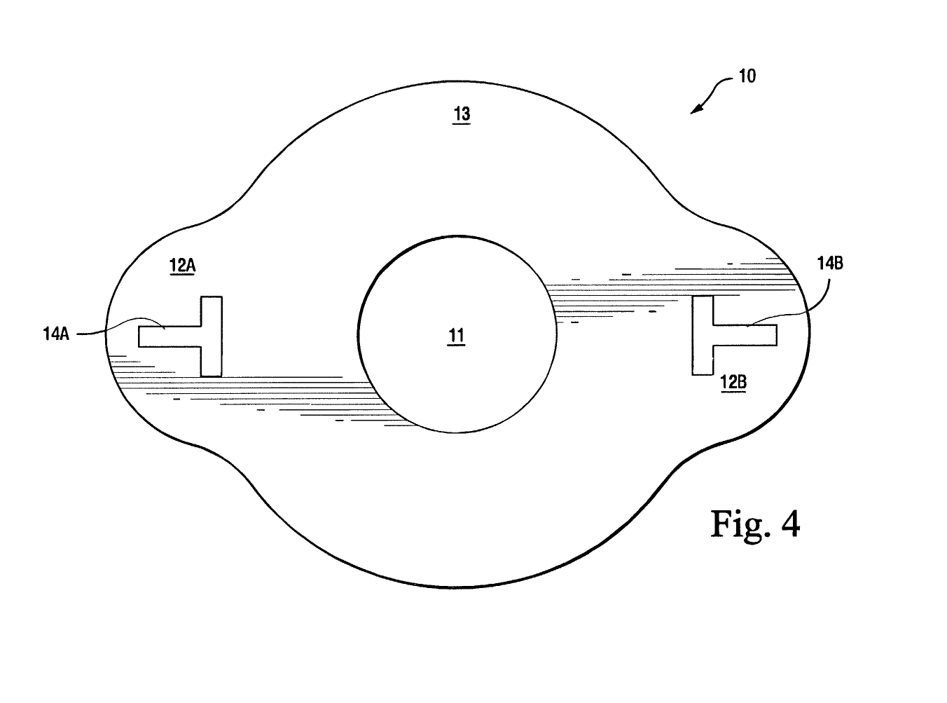

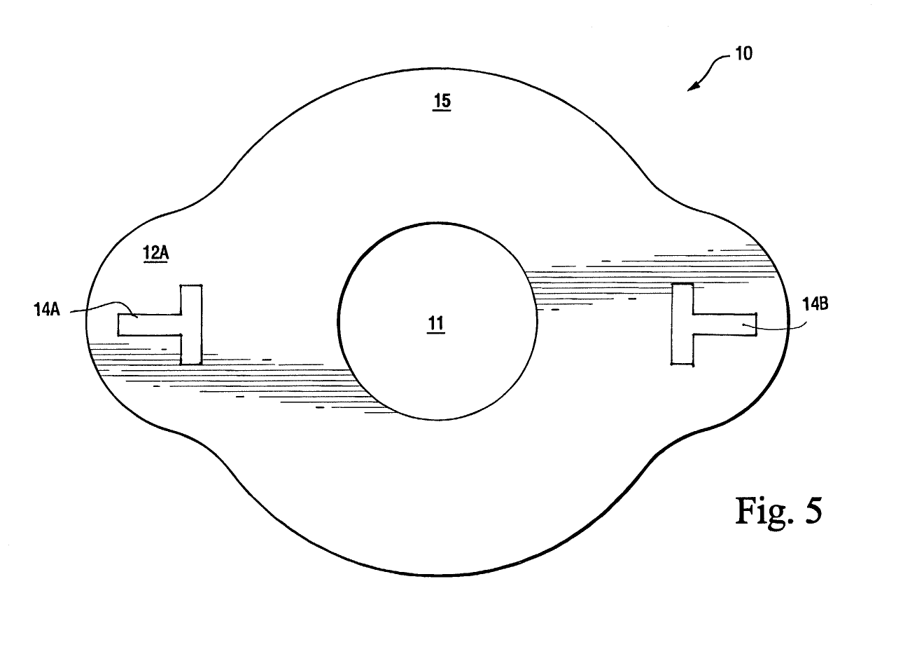

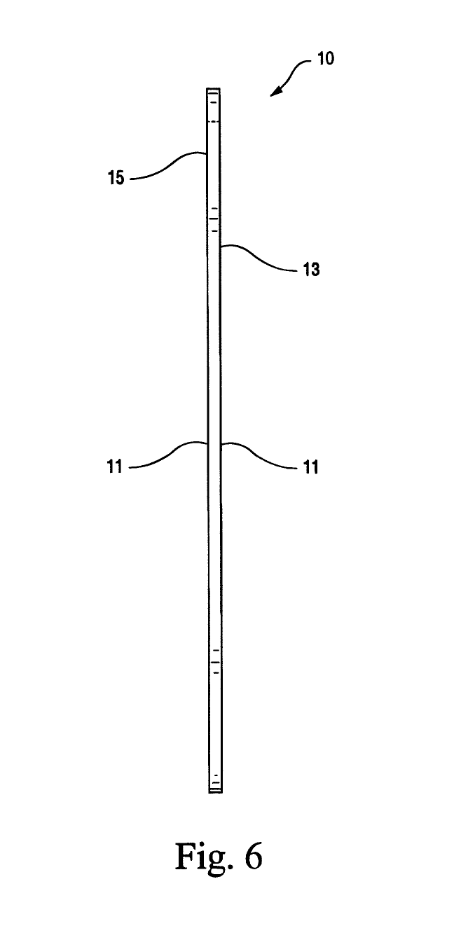

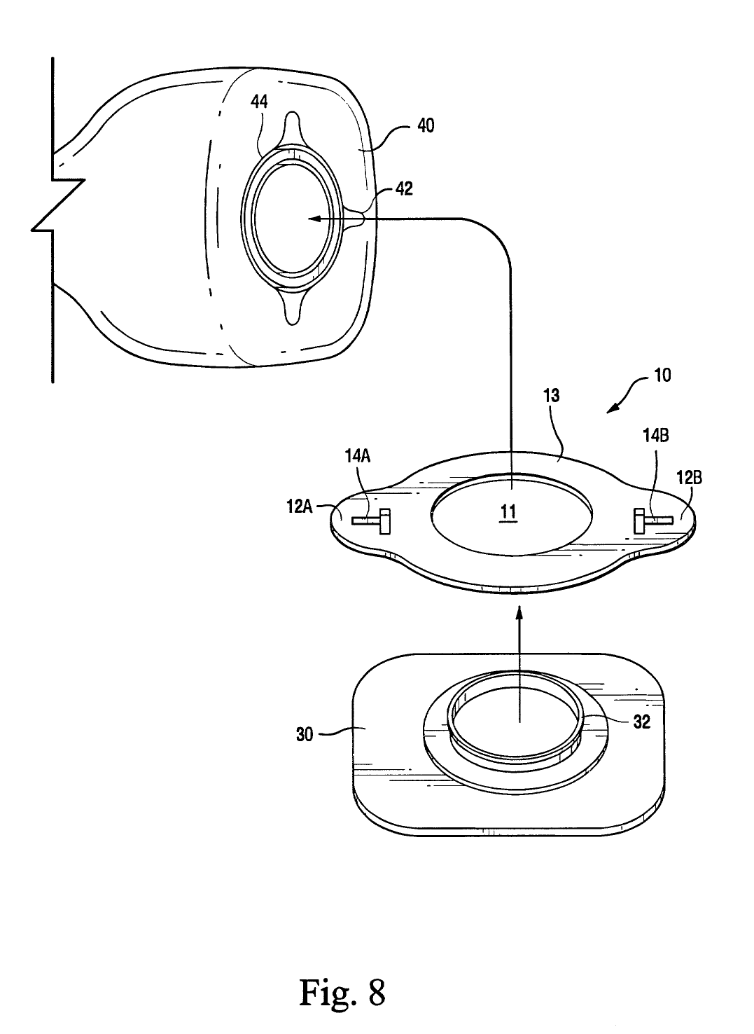

Referring to FIGS. 4, 5 and 6, shown are front, rear and side elevational views, respectively, of a second preferred embodiment of the invention. The second preferred embodiment comprises a flat plastic flange 10 with a central, circular aperture 11, and wings 12A and 12B, on which attachment structures are located. In the present invention, the attachment structures are preferably slots 14A and 14B. The slots 14A and 14B are preferably “T”-shaped and can engagingly receive the attachment mechanisms at the ends of an ostomy belt (not shown). The attachment mechanisms (which are conventionally tabs) at the ends of a conventional ostomy belt (not shown) can be received though the top of the “T”-shaped slot (14A and 14B), and slid along the slot to secure the belt. The aperture 11 has a diameter preferably slightly larger than the outer ridge 44 of the conventional double ridge adapter 42 of the ostomy pouch 40 shown in FIG. 8. Referring again to FIG. 8, in use, the single ridge adapter 32 of the wafer 30 engagingly interlocks with the double ridge adapter 42 of the ostomy pouch 40 through the aperture 11 of the flange 10. The front 13 of the flange 10 faces the ostomy pouch 40 and the rear 15 of the flange 10 faces the wafer 30.

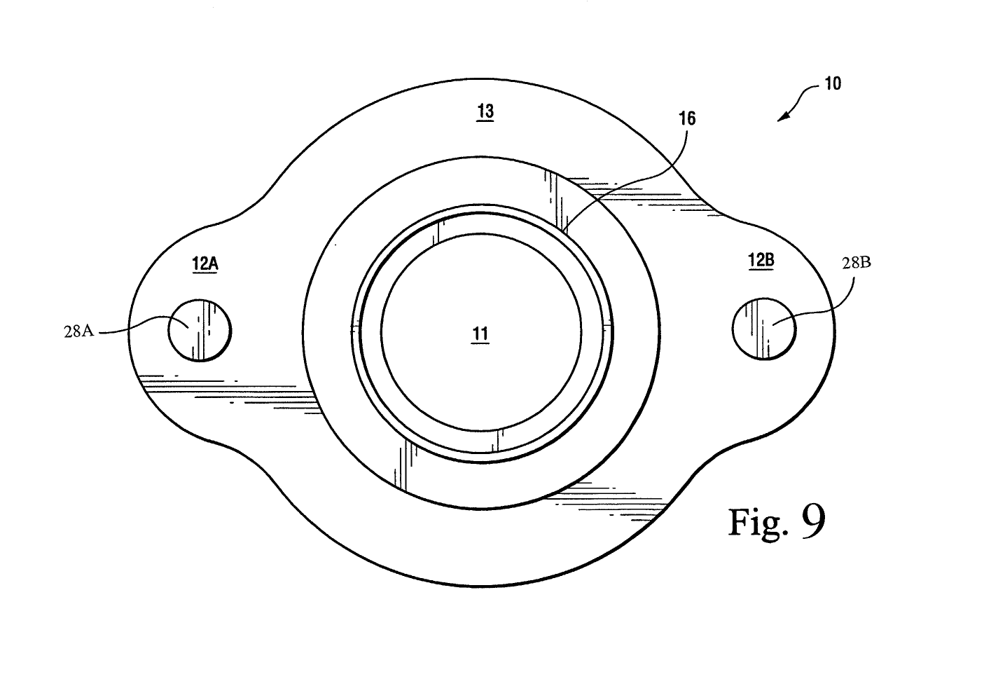

In alternative embodiments of the present invention, alternative attachment structures can be substituted for the slots 14A and 14B. For example, referring to FIG. 9, raised knobs 28A and 28B can be substituted for the slots 14A and 14B on the wings 12A and 12B of the present invention. Although FIG. 9 depicts the knobs 28A and 28B on an alternative embodiment of the first preferred embodiment of the present invention, it is understood that the knobs 28A and 29B can also be mounted on the wings 12A and 12B of an alternative embodiment of the second presently preferred embodiment of the present invention. In addition, tabs, hooks, buttons and hook and loop fasteners can be substituted as alternative attachment structures.

While this invention has been described with reference to ostomies, it may be used in connection with any surgical procedure that provides a stoma in the abdominal wall for discharging or receiving any substance without departing from the spirit and scope of the invention. Accordingly, no limitations shall be implied or inferred in the scope of this patent, except as specifically and explicitly set forth in the attached claims.

What is claimed is:

1. A device for holding a wafer having a circular single ridge over a stoma in a patient's skin, and for attaching an ostomy pouch having a circular, double ridge with an outer diameter to said wafer, said double ridge interlockable with said single ridge, comprising:
an elongated flange having a central, circular aperture slightly larger in diameter than said outer diameter;
a wing attached to each of two portions of said flange on approximately opposing sides of said aperture, each of said wings having an attachment structure thereon;
whereby a belt having an attachment mechanism complementary to said attachment structures can be attached to said attachment structures and said belt can be fastened around a user of said ostomy pouch to retain said wafer over said stoma;
wherein said wafer's single ridge and said ostomy pouch's double ridges can be interlocked through said central, circular aperture.

2. A device according to claim 1, wherein said flange is made of a pliable material.

3. A device according to claim 1, wherein said pliable material comprises plastic.

4. A device according to claim 1, wherein said wings are integrally formed with said flange.

5. A device for holding a wafer, having a front side and a back side and a circular single ridge, over a stoma in a patient's skin, and for attaching an ostomy pouch having a circular, double ridge with an outer diameter to said wafer, comprising:
an elongated flange having a central, circular aperture;
a circular single ridge adapter, complementary to and interlockable with said ostomy pouch's double ridge adapter, mounted on said front side of said flange;
a circular double ridge adapter mounted on said back side of said flange complementary to and interlockable with said wafer;
a wing attached to each of two portions of said flange on approximately opposing sides of said aperture, each of said wings having an attachment structure thereon;
whereby a belt having an attachment mechanism complementary to said attachment structures can be attached to said attachment structures and said belt can be fastened around a user of said ostomy pouch to retain said wafer over said stoma.

6. A device according to claim 5, wherein said flange is made of a pliable material.

7. A device according to claim 5, wherein said pliable material comprises plastic.

8. A device according to claim 5, wherein said wings are integrally formed with said flange.

9. A device for holding a wafer over a stoma and for attaching an ostomy pouch, comprising:
a flange adapted to be interposed between and sealingly engaged with both of said wafer and said ostomy pouch,;
attachment structures located on said flange;
whereby a belt having an attachment mechanism complementary to said attachment structures can be attached to said attachment structures and said belt can be fastened around a user of said ostomy pouch to retain said wafer over said stoma.

10. A device according to claim 1, wherein said attachment structures are selected from the group consisting of tabs, slots, hooks, buttons, and hook and loop fasteners.
